(19) United States
(12) Patent Application Publication
(10) Pub. No.: US 2012/0065122 A1
(43) Pub. Date: Mar. 15, 2012

(54) Pharmaceutical Composition and Method for Modulating Slow Myosin

(75) Inventors: Show-Li Chen, Taipei (TW); Hsin-Hsiung Chen, Taipei (TW); Szu-Wei Chang, Taipei (TW); Jim Pan, Taipei (TW); Kuan-Liang Lin, Taipei (TW)

(73) Assignee: NATIONAL TAIWAN UNIVERSITY, Taipei (TW)

(21) Appl. No.: 12/882,546

(22) Filed: Sep. 15, 2010

(51) Int. Cl. A61K 38/00

(52) U.S. Cl. 514/1.1

(57) ABSTRACT

The present invention directed to a method for modulating the expression level of slow myosin comprising administering to a subject in need thereof a therapeutically effective amount of nuclear receptor interaction protein (NRIP) modulator and calmodulin, and a pharmaceutically acceptable carrier.

### FIELD OF THE INVENTION

This present invention relates to a method for modulating the expression level of slow myosin. The method treats skeletal muscle dystrophy.

### DESCRIPTION OF PRIOR ART

The muscular dystrophies are a group of clinically and genetically heterogeneous disorders of the skeletal muscle inherited in either autosomal dominant or recessive fashion. Muscular dystrophies are characterized clinically by progressive muscle weakness predominantly in the pelvic and shoulder-girdle muscles, serum creatine kinase (SCK) elevation, normal intelligence and great variability, ranging from severe forms with onset in the first decade and rapid progression to milder forms with later onset and a slower course (Tsai, T. C. et al, J. Biol. Chem., 2005, 280, 20000-20009). The diagnosis of muscular dystrophies can be excluded by the finding of severely abnormal dystrophin staining on muscle biopsies. Although analysis of the defective proteins has shed some light onto their functions implicated in the etiology of muscular dystrophies, our understanding of the molecular mechanisms underlying muscular dystrophy remains incomplete.

Skeletal muscles are a mosaic of slow and fast twitch myofibers. Calcium (Ca⁺²) plays a key role in skeletal muscle contraction both in slow and fast fibers and regulates myosin heavy chain isoforms' gene expression. Now slow myosin fiber is clearly reportedly regulated by the increased intracellular Ca⁺². Additionally, testosterone increases the intracellular Ca⁺² level. Nuclear receptor interaction protein (NRIP) is a transcription cofactor, it contains 860 amino acids and seven copies of WD40 domains, and its expression is restricted to the cell nucleus. NRIP is an androgen receptor (AR)-interacting protein to enhance AR-mediated gene expression, it plays a feed-forward role in enhancing the AR-driven NRIP promoter activity via stabilization of the AR protein (Pei-Hong Chen et al, Nucleic Acids Research, 2008, Vol. 36, No. 1 51-66). NRIP enhances transcriptional activity of either AR or GR (glucocorticoid receptors) via ligand-dependent interactions (Tsai, T. C. et al, J. Biol. Chem., 2005, 280, 20000-20009).

In the recent report, the clinical gene expression profiles of muscular dystrophy patients lack NRIP gene expression by microarray assay. According to the analysis of differentially expressed genes between relative normal and dystrophic muscles from the same Limb-girdle muscular dystrophy (LGMD) patient, NRIP expression pattern was down-regulated in the muscular dystrophy patient (Yong Zhang et al, Journal of Translational Medicine, 2006, 4:53). However, the relation of NRIP caused muscular dystrophy needs to be further investigated.

### BRIEF DESCRIPTION OF THE DRAWINGS

FIG. 1. NRIP binds calmodulin in vivo and in vitro.

FIG. 1A. The IQ domain of NRIP locates on A.A. 676 to 706. The arrows indicate the highly conserved positions of amino acid compared with the other proteins containing IQ domain reported previously. The internal IQ-deleted mutant form of NRIP was generated by site-directed mutagenesis; and named NRIPΔIQ.

FIG. 1B. NRIP interacts with Ca⁺²/CaM in vitro. The NRIP proteins from in vitro translation (upper panel) or bacterially expressed (His-NRIP, lower panel) were incubated with CaM-agarose in the buffer containing calcium ions or EGTA. The proteins bound to CaM were then eluted by using EGTA-containing buffer and analyzed with anti-NRIP antibody. The data indicate NRIP bounds to CaM in the presence of calcium.

FIG. 1C. IQ domain of NRIP is responsible for Ca⁺²/CaM binding. The equal amounts of in vitro translated wt NRIP and IQ-deleted NRIP proteins of NRIPΔIQ were incubated with CaM-agarose. The CaM-binding proteins were then analyzed by western-blotting with anti-NRIP antibody.

FIG. 1D. NRIP interacts with Ca⁺²/CaM in vivo. The 293T cells were transiently co-transfected with NRIP-FLAG and CaM conjugated with EGFP expression plasmids. After 48 h, the cell lysates were collected and immunoprecipitated with anti-FLAG or anti-EGFP for NRIP and CaM, respectively. The immunoprecipitated proteins were then subjected to western-blotting with antibodies indicated.

FIG. 2. Generation of NRIP knockout mice.

FIG. 2A. Schematic illustration of genomic structure of the NRIP wild-type, NRIP flox, and NRIP-deleted alleles.

FIG. 2B. Southern blot hybridization of mouse tail genomic DNA isolated from wild-type (+/+) and heterozygous (+/−) offspring of heterozygous intercross. After restriction enzyme Sca I digestion and DNA denaturation, the genomic DNA was hybridized by 5′ flanking probe designed on NRIP intron 1 region. The wild-type allele represents a band on the size of 13.27 kb and the NRIP knockout allele represents a band on the size of 11.3 kb.

FIG. 2C. Genome typing of mouse tail DNA from wild-type (+/+), heterozygous (+/−) and homozygous(−/−) offspring by PCR analysis. The result showed a targeted product of 0.7 kb detected by AU-XD primers, and a wild-type product of 0.6 kb detected by KU-XD primers (*: nonspecific band).

FIG. 2D. Expression of NRIP mRNA level in NRIP knockout mice by RT-PCR analysis. The upper panel shows the schematic illustration of the designed primers to detect the deletion of NRIP exon 2; the lower panel shows RT-PCR analysis of NRIP mRNA isolated from testis, heart and skeletal muscle of wild-type(WT) and knockout(KO) offspring. β-actin or GAPDH was examined as a loading control.

FIG. 2E. Expression of mouse NRIP protein in wild-type (WT) and knockout (KO) adult tissues. Following tissue dissection and protein extraction, expression of NRIP was analyzed by Western blot with primary NRIP antibody. The size of NRIP protein was examined by knockdown of NRIP expression in LNCap human prostate cancer cell line (as a positive control). GAPDH was examined as a loading control. The left panel shows the expression of NRIP in WT and KO skeletal muscle tissue; the right panel shows the expression of NRIP and androgen receptor (AR) in WT and KO testis tissue.

FIG. 3. Expression of NRIP and slow myosin in skeletal muscle tissues of adult male mice (Following the tissue dissection and protein extraction).

FIG. 3A. Western blot analysis of NRIP expression, using total protein (100 μg) from the hindlimb skeletal muscle tissues of adult (10-week) male mice.

FIG. 3B. Analysis of slow myosin (MHC7) expression in soleus and gastrocnomius (Gast.)

muscle tissues respectively.

The size of NRIP protein was examined by knockdown of NRIP expression in LNCap human prostate cancer cell line. The GAPDH serves as an internal control for protein loading.

FIG. 4. RNA expression of slow myosin in soleus muscle tissues. As described tissues from FIG. 3, RNA was extracted and analyzed for the gene expression of slow myosin (MHC7).

FIG. 5. Immunohistochemistry analysis of slow myosin expression in gastrocnomius skeletal muscle tissue of 12-week old NRIP+/+ and NRIP−/− mice. Following tissue dissection and paraffin embedding, the 4 μm sections were incubated with slow myosin primary antibody (MHC 7) for overnight and stained with 3,3′ Diaminobenzidine (DAB) chromogen. In wild-type mice (A and B), the slow myosin was expressed dispersedly in gastrocnomius tissue. In NRIP−/− mice (C and D), the slow myosin was less expressed in this tissue. (magnification: A and C ×100; B and D ×200). Arrow mark: slow myosin.

### SUMMARY OF THE INVENTION

The present invention relates to a method for modulating the expression level of slow myosin comprising administering to a subject in need thereof a therapeutically effective amount of nuclear receptor interaction protein (NRIP) modulator and calmodulin, and a pharmaceutically acceptable carrier.

### DETAILED DESCRIPTION OF THE INVENTION

The present invention showed that NRIP is Ca⁺²-dependent calmodulin binding protein. Moreover, preliminary results of the present invention from NRIP knock out mice model demonstrates the slow myosin protein and RNA expression are declined in NRIP KO mice. Therefore, NRIP may be involved in skeletal muscle development and be a diagnosis marker and therapeutic target of muscular dystrophy.

The present invention directed to a method for modulating the expression level of slow myosin comprising administering to a subject in need thereof a therapeutically effective amount of nuclear receptor interaction protein (NRIP) modulator and calmodulin, and a pharmaceutically acceptable carrier. The nuclear receptor interaction protein (NRIP) binds with the calmodulin. And the expression level of slow myosin is protein expression level or RNA expression level. The pharmaceutical composition of the present invention treats skeletal muscle dystrophy.

### EXAMPLES

The examples below are non-limiting and are merely representative of various aspects and features of the present invention.

### Example 1

### NRIP Binds Calmodulin In Vitro and In Vivo

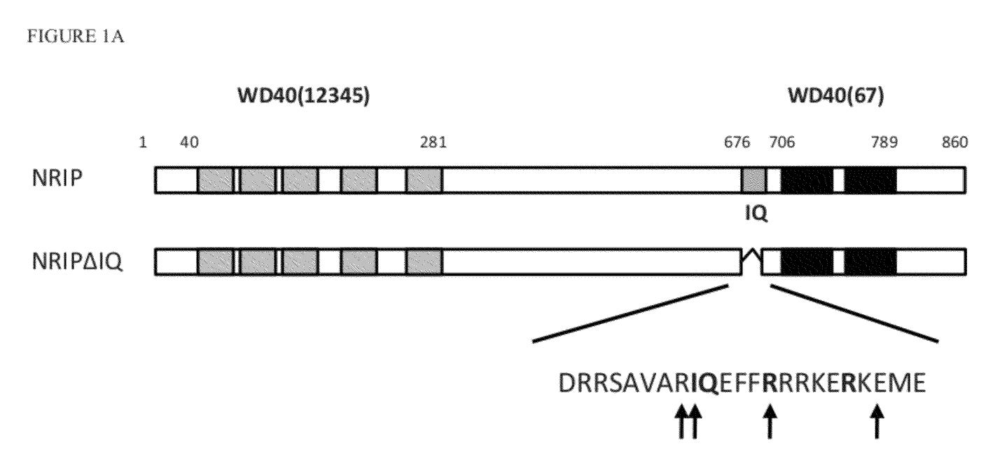

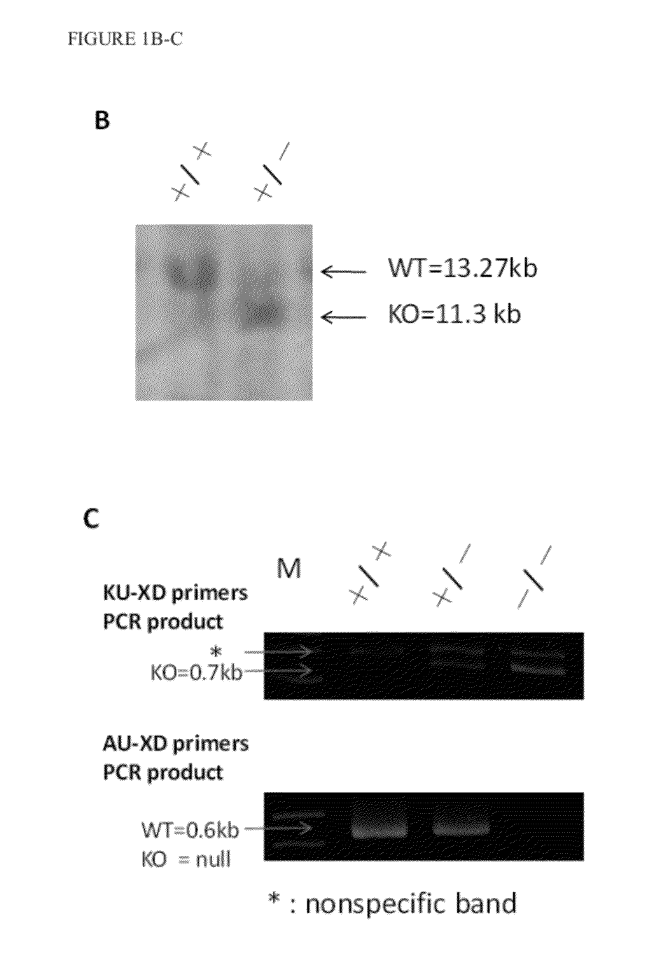

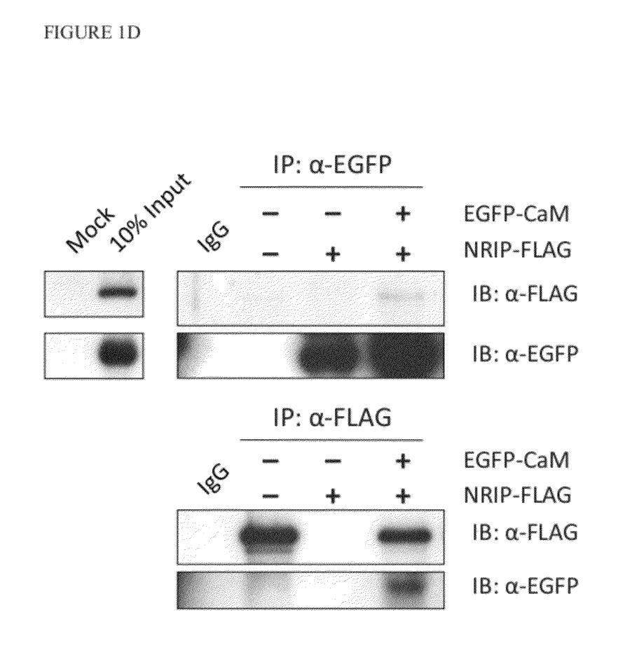

The wild-type NRIP proteins and IQ-deleted NRIP proteins from in vitro translation or bacterially expressed His-NRIP were incubated with CaM-agarose. The proteins bound to CaM were then eluted by using EGTA-containing buffer and analyzed with anti-NRIP antibody. These data indicate NRIP bounds to CaM in the presence of calcium (FIG. 1B and FIG. 1C). To test the NRIP that could interact with CaM in vivo, the 293T cells were transiently co-transfected with NRIP-FLAG and CaM conjugated with EGFP expression plasmids. After 48 h, the cell lysates immunoprecipitated with anti-FLAG or anti-EGFP for NRIP and CaM, respectively and then analyzed with immunoblot (FIG. 1D). The results showed that NRIP interacts with CaM.

### Example 2

### Generation of NRIP Knockout Mice

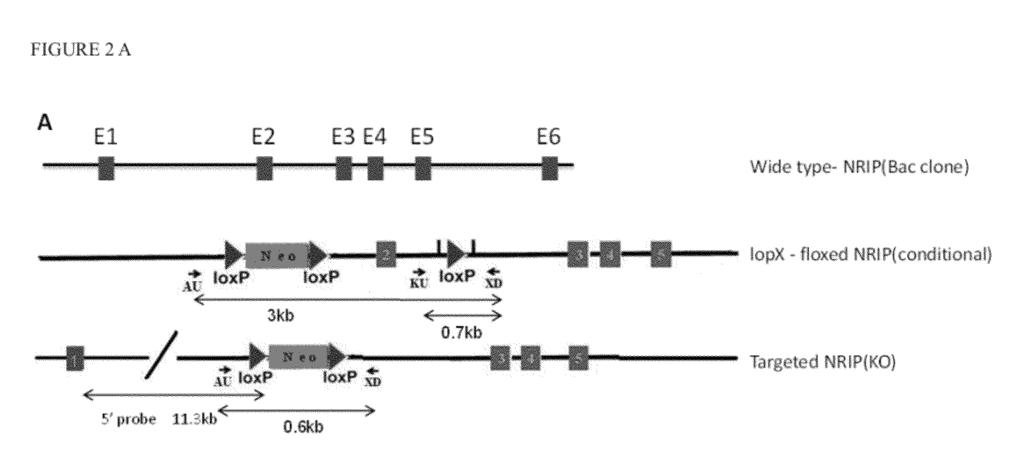

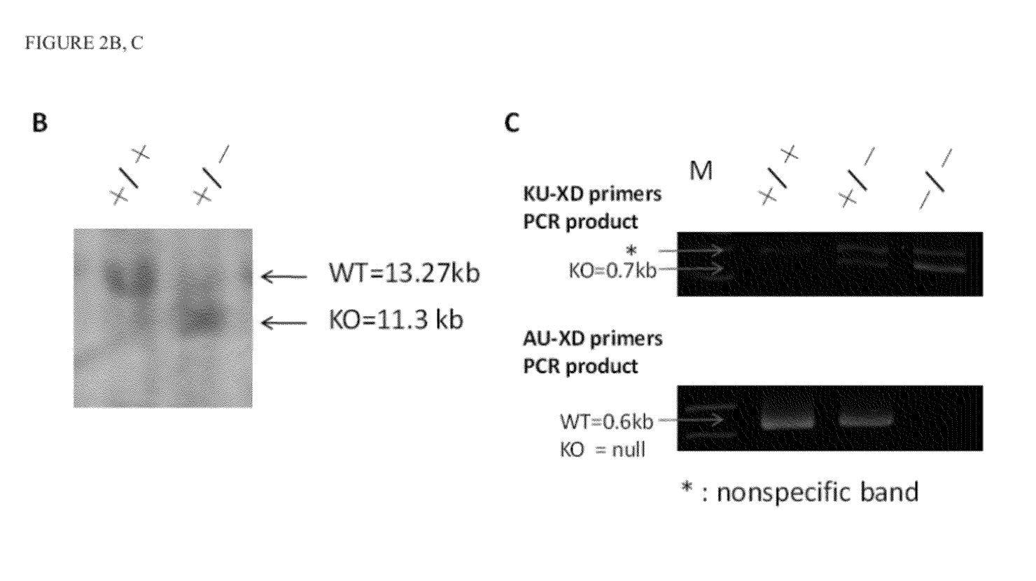

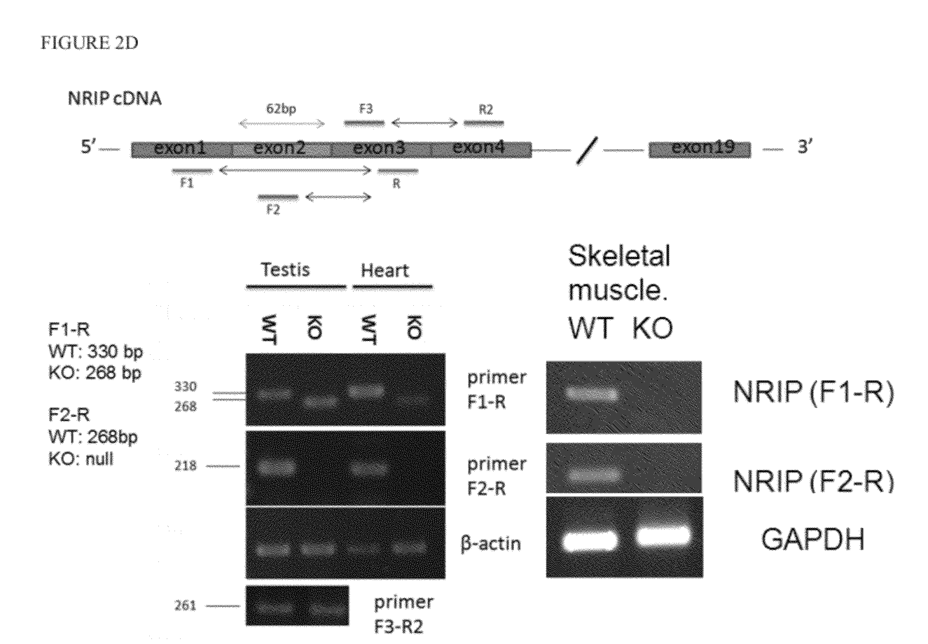

The loxP-floxed NRIP conventional knockout mice were suitable for investigating the role of NRIP in skeletal muscle development. The NRIP exon2 was deleted after loxP site recombination (FIG. 2A). The genome NRIP deletion was confirm by Southern blot (FIG. 2B) and mouse tail genometyping (FIG. 2C), respectively. The present invention also detected the expression of NRIP mRNA in the testis, heart and skeletal muscle tissues. The results showed that the exon2 deleted NRIP was detected by the designed F 1-R primers and was not detected by the designed F2-R primers (FIG. 2D). The expression of NRIP protein in testis and skeletal muscle tissue was also performed by Western blot, in this result, the NRIP was expressed in the wild-type mouse testis and skeletal muscle tissues but not in NRIP-null mouse testis and skeletal muscle tissues (FIG. 2E).

### Example 3

### Expression of NRIP and Slow Myosin in Skeletal Muscle of Adult Male Mice

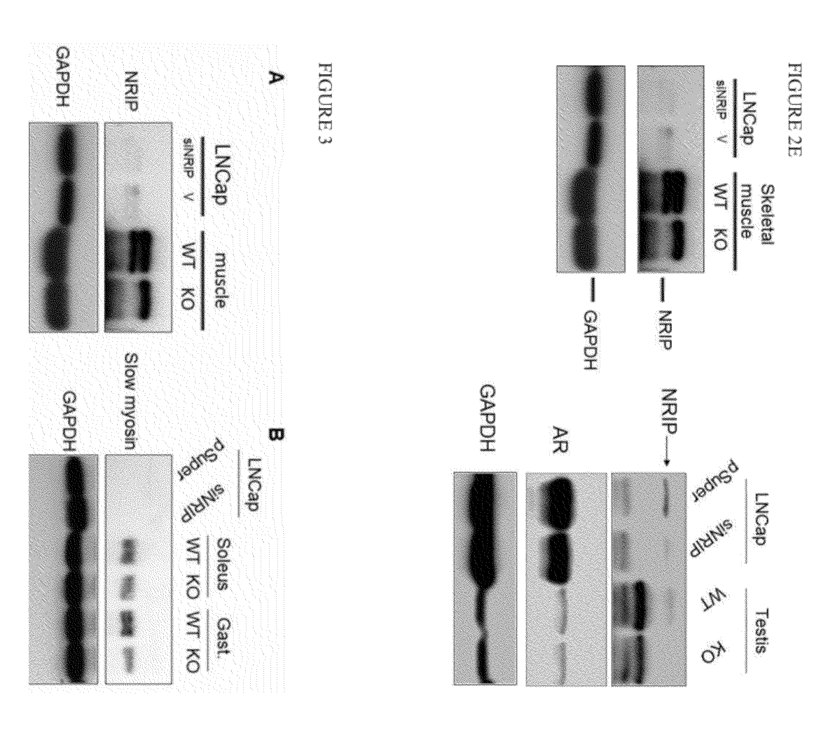

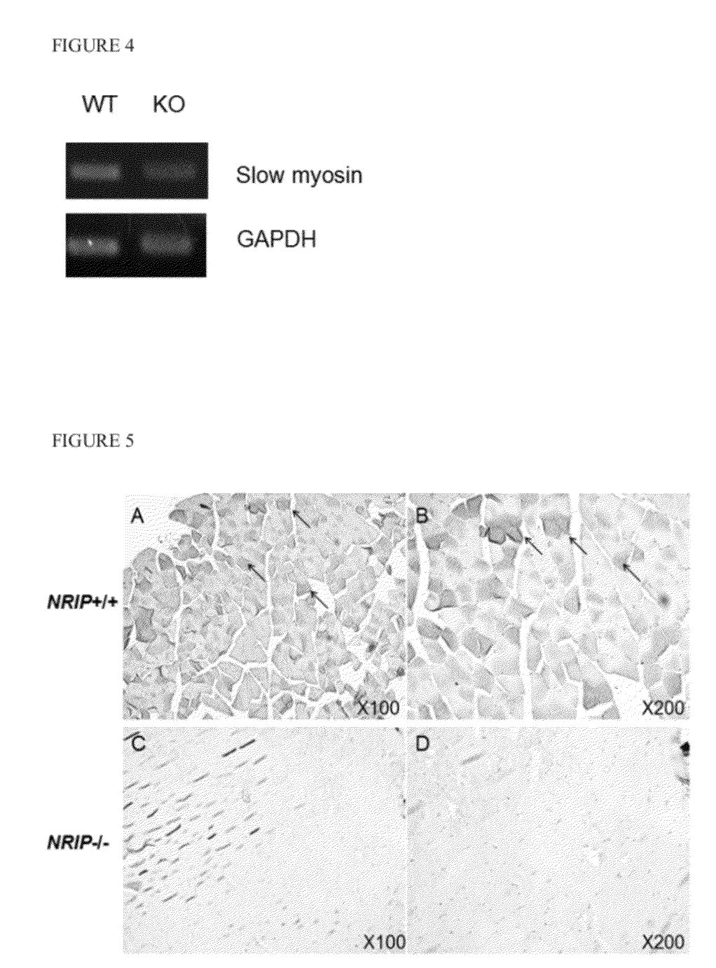

The previous results showed that the NRIP can bind to CaM. Besides, the expression of slow myosin was controlled by the Ca²⁺/CaM signaling pathway. Hence, the present invention next investigated the expression of slow myosin in NRIP wild-type and null mice. The present invention dissected the mouse soleus and gastrocnomius muscle tissue and the protein was extracted by RIPA buffer. The slow myosin and NRIP protein expression was performed by the Western blot. The results showed that the expression of slow myosin was decreased in NRIP null mice (FIG. 3B). The expression of NRIP mRNA was also decreased in NRIP null mice (FIG. 4). Moreover, the present invention also examined the expression of NRIP protein in gastrocnomius skeletal muscle tissues by IHC analysis, the result showed that the expression of NRIP was dromatically decreased in NRIP null mice (FIG. 5).

While the invention has been described and exemplified in sufficient detail for those skilled in this art to make and use it, various alternatives, modifications, and improvements should be apparent without departing from the spirit and scope of the invention.

What is claimed is:

1. A method for modulating the expression level of slow myosin comprising administering to a subject in need thereof a therapeutically effective amount of nuclear receptor interaction protein (NRIP) modulator and calmodulin, and a pharmaceutically acceptable carrier.

2. The method of claim 1, wherein the nuclear receptor interaction protein (NRIP) binds with the calmodulin.

3. The method of claim 1, wherein the expression level of slow myosin is protein expression level.

4. The method of claim 1, wherein the expression level of slow myosin is RNA expression level.

5. The method of claim 1, which treats skeletal muscle dystrophy.
